(19) United States
(12) Patent Application Publication
(10) Pub. No.: US 2002/0051795 A1
(43) Pub. Date: May 2, 2002

(54) ASCORBIC ACID AS AN ADJUVANT IN THE REMOVAL OF VASCULAR OCCLUSIONS

(76) Inventor: JURGEN F. PASCOE, POHLHEIM (DE)

Correspondence Address: COLLARD & ROE, P.C., 1077 NORTHERN BOULEVARD, ROSLYN, NY, 11576, US

(21) Appl. No.: 09/381,089

(22) PCT Filed: Jan. 30, 1998
(86) PCT No.: PCT/EP98/00486

(30) Foreign Application Priority Data
Mar. 15, 1997 (DE) 197 10 916.0

(51) Int. Cl. A61K 9/00

(52) U.S. Cl. 424/400

(57) ABSTRACT

The invention relates to the use of ascorbic acid or one of its physiologically tolerable salts for producing a medicament which contains 5 to 50 g of ascorbic acid and is used as an adjuvant in the removal of occular occlusions. The invention also relates to combined preparations which contain large-dose ascorbic acid together with ±-tocopherol compounds and/or thrombolytically active compounds.

The present invention relates to the use of ascorbic acid or one of its physiologically tolerable salts for production of pharmaceutical drugs that can be used to advantage in eliminating vascular occlusions.

It is known that a vascular occlusion can lead to life-threatening conditions or the loss of a limb due to the formation of a thrombus in a coronary vessel or other organs of the body, e.g., the extremities. The quickest possible elimination of the thrombus and recanalization to restore unhindered blood flow, re-perfusion, are necessary to prevent necrosis in order to prevent permanent damage or even death.

Several invasive methods are available to restore arterial blood flow through the vessels, namely thrombolysis, percutaneous transluminal angioplasty and bypass surgery.

Each of the treatment methods mentioned above has both advantages and disadvantages. Although elimination of a vascular occlusion by intravenous administration of a thrombolytic substance is a gentle method, it frequently does not lead to opening of the occluded vessel quickly enough. In addition, unwanted bleeding may also occur as a side effect.

Percutaneous transluminal angioplasty with the help of a balloon catheter leads to rapid recanalization of the blood vessel, but it can also lead to damage due to distension of the blood vessel, and it does not offer any guarantee that the resulting opening of the vessel will be maintained for a long period of time. Instead, various experiences indicate that the lumen of a vessel opened by a balloon catheter will often close again after a short time.

In bypass surgery, the blood vessel must be clamped off for the duration of the operation, with the blood flow being released again after insertion of the bypass. However, damage occurs here during re-perfusion due to the increased occurrence of free oxygen radicals; this is also the reason for the heretofore unsatisfactory results of angioplastic vasodilation by means of a balloon catheter. Thus, it has been demonstrated in animal experiments in particular that tissue damage occurring when restoring circulation may lead to arrhythmias or even cardiac arrest in recanalization of the coronary vessels. The assumption that these postoperative complications in re-perfusion of ischemic myocardium might be caused by a significantly increased level of free oxygen radicals has already been the subject of several scientific studies [1-3]. As a result, it can be concluded that the formation of free oxygen radicals is evidently closely related to the damage occurring due to re-perfusion. Free radicals react with many elements of the cell structure such as biomembranes, liposomes and mitochondria, thereby destroying them. This is the main reason for the re-perfusion damage.

Substances with an antioxidant effect such as vitamin C and vitamin E are normally present in the human body and can capture the quantities of oxygen radicals that usually occur. The quantities of these substances that must be present to neutralize the unusually large quantities of oxygen radicals occurring during or after ischemia have not yet been determined conclusively.

As in bypass surgery, free oxygen radicals are also formed in large quantities in angioplastic vasodilation by means of a balloon catheter. In addition, scientific studies indicate that percutaneous transluminal angioplasty performed using a balloon catheter often does not result in permanent opening of the vessel because the initial success is undone by a rapid stenosis of the vessel again. Free oxygen radicals, which are present in large quantities and are observed before, during and after the surgical procedure, are also responsible for this. Distension of the inside wall of the vascular lumen evidently leads to tissue damage which is not only compensated by an accelerated and increased growth of the internal vascular wall (intima) but also results in increased deposition of oxidation products of low-density lipoproteins.

Therefore, the object is to make available a pharmaceutical drug that is capable of suppressing the complications that occur during the elimination of vascular occlusions due to the formation of unusually large quantities of free oxygen radicals.

This object is achieved by a pharmaceutical drug which contains ascorbic acid or one of its physiologically tolerable salts in a parenteral dosage form and is administered before, during and/or after a thrombolytic treatment, bypass surgery or percutaneous transluminal angioplasty. The ascorbic acid or its salt must be administered in an unusually high concentration, namely in an amount of 50 to 500 mg/kg body weight per day, because only in this way are sufficiently high concentrations of the active ingredient achieved in the body to capture all the oxygen radicals released in a surgical procedure.

It is surprising that the correlation found in animal experiments between the formation of large quantities of oxygen radicals and the occurrence of tissue damage and arrhythmias or even cardiac arrest, the causes of which are still not fully understood, can evidently also be observed in the human body. In particular, it was not to be expected that the results of eliminating a vascular occlusion in humans could be influenced by using a pharmaceutical drug containing ascorbic acid. It should be remembered that apart from humans and other primates, guinea pigs, some birds and fish, all other animals, i.e., even the experimental animals used in earlier medical studies such as swine, dogs or rats have the ability to synthesize ascorbic acid themselves. Therefore, they generally have sufficiently large quantities of ascorbic acid in their bodies to neutralize harmful oxygen radicals. If there are harmful effects due to free oxygen radicals in such experimental animals despite the presence of natural ascorbic acid, then ascorbic acid would not fundamentally be expected to be capable of rendering these oxygen radicals harmless to a sufficient extent. However, if oxygen radicals cannot be captured sufficiently even in those experimental animals that synthesize ascorbic acid themselves, then ascorbic acid could not be expected to achieve this object in humans.

It has now been found that the favorable effects of ascorbic acid and its salts as an adjuvant in eliminating vascular occlusions in humans can be further enhanced if the ascorbic acid is administered in combination with another antioxidant vitamin, vitamin E or another substance from the alpha-tocopherol group. Since the tocopherols are fat-soluble and vitamin C is water-soluble, the two vitamins complement one another optimally when they are used according to this invention, not in mixture but as individual components of a packaging unit for simultaneous, separate or chronologically staggered parenteral administration.

It had already been shown on swine heart that combined administration of vitamins C and E can improve endothelial functions in hypercholesterolemia and can prevent deposition of lipids in animal models created for detection of arteriosclerosis [4]. However, it was completely unknown whether such a combination of ascorbic acid and tocopherols can protect the myocardium and other human tissue from the damage occurring in the course of a thrombolytic procedure, bypass surgery or percutaneous transluminal angioplasty.

It has surprisingly been found, however, that the tissue damage caused by a thrombolytic treatment, by bypass surgery or by percutaneous transluminal angioplasty is influenced in a remarkably favorable manner according to this invention.

One explanation for the synergistic effects of a combination therapy with ascorbic acid and tocopherols as adjuvants in eliminating vascular occlusions might be that the water-soluble ascorbic acid can carry out its function as a scavenger of free oxygen radicals only in the aqueous intracellular and extracellular area. However, the fat-soluble alpha-tocopherol also acts in the lipophilic areas of the cell tissue and can capture free oxygen radicals in areas where ascorbic acid cannot. This is important because peroxidation of polyunsaturated fatty acids in the lipids of the cell membranes is thought to be responsible for the damage caused by free oxygen radicals. Vitamin E in the presence of ascorbic acid can effectively ensure protection of polyunsaturated fatty acids because oxidized alpha-tocopherol is reduced again by vitamin C and then can again manifest its effect of protecting polyunsaturated fatty acids from oxidation by free oxygen radicals.

The present invention also relates to a combination product which includes, separately from one another in one packaging unit, a thrombolytic compound such as streptokinase, urokinase, hirudine, heparin or antithrombin III in a ready-to-use dosage form and a ready-to-use pharmaceutical formulation containing ascorbic acid or one of its physiologically tolerable salts plus optionally also a ready-to-use tocopherol product. These preparations are used as a parental dosage form for concomitant, separate or chronologically staggered treatment to eliminate vascular occlusions.

Large quantities of free oxygen radicals are also formed in thrombolytic elimination of vascular occlusions by re-perfusion, causing biochemical oxidation reactions which can lead to severe tissue damage, ventricular fibrillations or even cardiac arrest, so there is also a need in this case as well to prevent the feared complications by administering large quantities of ascorbic acid or one of its salts. By concomitant, separate or chronologically staggered parenteral administration of ascorbic acid or one of its salts together with a thrombolytic compound suitable for parenteral administration plus optionally an alpha-tocopherol compound that can be administered parenterally, it is possible to prevent in an especially effective manner the complications that would otherwise occur in thrombolytic removal of vascular occlusions. All the pharmaceutical products required for this treatment are made available to physicians in a single packaging unit according to this invention, so that they have on hand all the required medications available immediately for therapy, where success depends overwhelmingly on the fastest possible elimination of the vascular occlusion, and they can perform the required treatment measures continuously one after the other.

Treatment of a patient threatened by a vascular occlusion is administered, for example, by first giving the patient streptokinase (1.0×106 units) or heparin in a dose of 2.5 mg/kg over a period of four hours in the form of an infusion over a period of 30 minutes. At the same time, the patient receives over a period of four hours an infusion of 70 ml of a 15% ascorbic acid solution diluted with physiological saline solution and adjusted to a pH of 6.0 to 7.0. The patient's condition is monitored continuously by an electrocardiogram.

A group of 20 patients at risk for a vascular occlusion were treated by the procedure described above and compared with an equal number of patients receiving no ascorbic acid infusion during the operation but otherwise the same therapy. It was found that significantly fewer complications occurred in the patient group treated with the ascorbic acid infusion during the operation and during the subsequent two-week observation phase.

### SUMMARY OF THE LITERATURE

[1] E. D. Grech, N. J. F. Dodd, C. M. Bellamy et al.: “Free radical generation during angioplasty reperfusion for acute myocardial infarction,” The Lancet, 341, 991-992 (1993)

[2] E. D. Grech, C. M. Bellamy, M. J. Jackson et al.: “Free radical activity after primary coronary angioplasty in acute myocardial infarction,” American Heart Journal, Vol. 127, No. 6, 1443-1449 (1994)

[3] R. Bolli, M. O. Jeroudi, B. S. Patel, O. l. Aroumo et al.: “Marked reduction of free radical generation and contractile dysfunction by antioxidant therapy begun at the time of re-perfusion,” Circulation Research, 65, 607-622 (1989)

[4] G. L. Nunes, D. S. Sgoutas, S. S. Redden et al.: “Combination of vitamins C and E alters the response to coronary balloon injury in the pig,” Arteriosclerosis, Thrombosis, and Vascular Biology, Vol. 15, No. 1, 156-165 (1995)

1. Use of ascorbic acid or one of its physiologically tolerable salts for production of a pharmaceutical drug suitable for parenteral administration, characterized in that the ascorbic acid or its salt is administered in an amount of 50 to 500 mg/kg body weight per day as an adjuvant in the treatment of diseases caused by pathologically elevated formation of free radicals.

2. Use according to claim 1, characterized in that the ascorbic acid or its salt is administered in an amount of 50 to 500 mg/kg body weight per day as an adjuvant in the elimination of vascular occlusions.

3. Use according to claim 2, characterized in that the pharmaceutical preparation is administered before, during or after a thrombolytic treatment, bypass surgery or percutaneous transluminal angioplasty.

4. Combination product, characterized in that it contains in one packaging unit, separately from one another
a) a ready-to-use pharmaceutical product suitable for parenteral administration and containing ascorbic acid or one of its physiologically tolerable salts and
b) a ready-to-use pharmaceutical product suitable for parenteral administration and containing an alpha-tocopherol compound,
that are used for concomitant, separate or chronologically staggered use as adjuvants in the elimination of vascular occlusions.

5. Combination product according to claim 4, characterized in that it contains in one packaging unit, separately from one another
a) a ready-to-use pharmaceutical product suitable for parenteral administration and containing more than 5 g of ascorbic acid or one of its physiologically tolerable salts and
b) a ready-to-use pharmaceutical product suitable for parenteral administration and containing 0.1 to 2 g of an alpha-tocopherol compound.

6. Combination product, characterized in that it contains in one packaging unit, separately from one another
a) a ready-to-use pharmaceutical product suitable for parenteral administration and containing ascorbic acid or one of its physiologically tolerable salts and
b) a ready-to-use pharmaceutical product suitable for parenteral administration and containing a thrombolytic compound plus optionally in addition
c) a ready-to-use pharmaceutical product suitable for parenteral administration and containing an alpha-tocopherol compound,
which are used for concomitant, separate or chronologically staggered use in elimination of vascular occlusions.

7. Combination product according to claim 6, characterized in that contains in one packaging unit, separately from one another
a) a ready-to-use pharmaceutical product suitable for parenteral administration and containing more than 5 g ascorbic acid or one of its physiologically tolerable salts and
b) a ready-to-use pharmaceutical product suitable for parenteral administration and containing a thrombolytic compound such as streptokinase, urokinase, hirudine, heparin or antithrombin as dry substances together with a suitable solvent in a suitable amount for elimination of a vascular occlusion plus optionally in addition
c) a ready-to-use pharmaceutical product suitable for parenteral administration and containing 0.1 to 2.0 [g] of an alpha-tocopherol compound.
